(19) United States
(12) Patent Application Publication
(10) Pub. No.: US 2017/0049993 A1
(43) Pub. Date: Feb. 23, 2017

(54) ECHOGENIC PROBE

(71) Applicant: COSMAN MEDICAL INC., BURLINGTON, MA (US)

(72) Inventors: Eric R. Cosman, Belmont, MA (US); Eric R. Cosman, Belmont, MA (US)

(21) Appl. No.: 13/966,958

(22) Filed: Aug. 14, 2013

Related U.S. Application Data
(60) Provisional application No. 61/683,190, filed on Aug. 14, 2012.

(51) Int. Cl. A61M 25/01; A61B 18/14; A61B 18/18

(52) U.S. Cl. CPC A61M 25/0105; A61B 18/18; A61B 18/1477

(57) ABSTRACT

Echogenic markers can be applied to probes such as medical needles, including radiofrequency cannulae, injection needles, biopsy needles, microwave antennae, and spinal needles, among others. For example, in certain embodiments, the probes may have a distal end, a proximal end, a shaft, and an echogenic feature in the form of one or more indentations on the shaft. In certain embodiments, the probes may have a first echogenic feature in the form of an indentation in a surface of the probe and a second echogenic feature in the form of a roughening of the surface of the probe.

### PRIORITY CLAIM

This application claims priority to U.S. Provisional Application No. 61/683,190, filed Aug. 14, 2012, which is incorporated by reference in its entirety.

### TECHNICAL FIELD OF THE INVENTION

The invention relates generally to probes used in medical procedures. The invention relates more specifically to means of enhancing the ultrasound image of probes used in medical procedures. The invention relates more specifically to field therapy.

### BACKGROUND OF THE INVENTION

The use of radiofrequency (RF) generators and electrodes to be applied to tissue for pain relief or functional modification is well known. For example, the RFG-3B RF lesion generator of Radionics, Inc., Burlington, Mass. and its associated electrodes enable electrode placement of the electrode near target tissue and heating of the target tissue by RF power dissipation of the RF signal output in the target tissue. For example, the G4 generator of Cosman Medical, Inc., Burlington, Mass. and its associated electrodes such as the Cosman CSK, and cannula such as the Cosman CC and RFK cannula, enable electrode placement of the electrode near target tissue and heating of the target tissue by RF power dissipation of the RF signal output in the target tissue. Temperature monitoring of the target tissue by a temperature sensor in the electrode can control the process. Heat lesions with target tissue temperatures of 60 to 95 degrees Celsius are common. Tissue dies by heating above about 45 degrees Celsius, so this process produces the RF heat lesion. RF generator output is also applied using a pulsed RF method, whereby RF output is applied to tissue intermittently such that tissue is exposed to high electrical fields and average tissue temperature are lower, for example 42 degrees Celsius or less.

RF generators and electrodes are used to treat pain, cancer, and other diseases. Examples are the equipment and applications of Cosman Medical, Inc., Burlington, Mass. such as the G4 radiofrequency generator, the CSK electrode, CC cannula, and DGP-PM ground pad. Related information is given in the paper by Cosman E R and Cosman B J, “Methods of Making Nervous System Lesions”, in Wilkins R H, Rengachary S (eds.); Neurosurgery, New York, McGraw Hill, Vol. 3, 2490-2498; and is hereby incorporated by reference in its entirety. Related information is given in the book chapter by Cosman E R Sr and Cosman E R Jr. entitled “Radiofrequency Lesions.”, in Andres M. Lozano, Philip L. Gildenberg, and Ronald R. Tasker, eds., Textbook of Stereotactic and Functional Neurosurgery (2nd Edition), 2009, and is hereby incorporated by reference in its entirety.

The Cosman CC cannula and RFK cannula, manufactured by Cosman Medical, Inc. in Burlington, Mass., include each an insulated cannula having a pointed metal shaft that is insulated except for an uninsulated electrode tip. The CC cannula has a straight shaft. The RFK cannula has a curved shaft; one advantage of a curved shaft is that it can facilitate maneuvering of the cannula's tip within tissue. Each cannula includes a removable stylet rod that occludes the inner lumen of the cannula's shaft, for instance during insertion of the cannula into solid tissue, and can be removed to allow for injection of fluids or insertion of instruments, like an electrode. The cannula has a hub at its proximal end having a luer fitting to accommodate a separate thermocouple (TC) electrode, for example the Cosman CSK electrode, Cosman TCD electrode, and Cosman TCN electrode, that can deliver electrical signal output such as RF voltage or stimulation to the uninsulated electrode tip. The Cosman CSK and TCD electrodes have a shaft that is stainless steel. The Cosman TCN electrode has a shaft that is Nitinol. Related information is given in Cosman Medical brochure “Four Electrode RF Generator”, brochure number 11682 rev A, copyright 2010, Cosman Medical, Inc., and is hereby incorporated by reference herein in its entirety. One limitation of the CC and RFK RF cannulae is that they do not include echogenic markers.

A paper by S N Goldberg et al. entitled “Hepatic Metastases: Percutaneous Radiofrequency Ablation with Cool-Tip Electrodes,” Radiology 2007, vol. 205, no. 2, pp. 367-373 describes various techniques and considerations relating to tissue ablation with RF electrodes have cooled electrode tips, and is incorporated herein by reference. The Cool-Tip Electrode of Radionics and Valley Lab, Inc. is a 16-gauge (or 1.6 millimeter diameter) electrode with partially insulated shaft and water-cooling channel inside its rigid, straight cannula shaft. The brochure from Radionics is hereby incorporated by reference in its entirety. The Cool-Tip Electrode is used for making large RF heat ablations of cancerous tumors, primarily in soft-tissue organs and bone. It has a closed trocar point that includes a metal plug that is welded to the metal tubing that is part of the electrode shaft. The distal end of the metal plug is sharpened to form a three sided, axially symmetric trocar. The distal end is a closed and sealed metal structure. The sharpened portion of the distal tip does not include the metal tubing itself, but rather the sharpened end of the metal plug that is welded to the metal tubing. This has the limitation that the shaft is not curved. This has the limitation that the shaft does not contain both echogenic markers and a curved tip. This has the limitation that it is not a hollow shaft covered in part by electrical insulation and having echogenic markers.

A paper by Rosenthal et al entitled “Percutaneous Radiofrequency Treatment of Osteoid Osteoma,” Seminars in Musculoskeletal Radiology, Vol. 1, No. 2, 1997 reports the treatment of a primary benign bone tumor using a percutaneously placed radiofrequency electrode, and is incorporated herein by reference.

Medical needles are used for epidural anesthesia, for example, for the introduction of catheters into the epidural space for the purpose of treating pain. Examples of epidural introducer needles include the tuohy needle, and the needle disclosed in U.S. Pat. No. 5,810,788 authored by Racz. Related information on epidural anesthesia and epidural needles is in “Epidural Lysis of Adhesions and Percutaneous Neuroplasty” by Gabor B. Racz, Miles R. Day, James E. Heavner, Jeffrey P. Smith, Jared Scott, Carl E. Noe, Laslo Nagy and Hana Ilner (2012), in the book “Pain Management—Current Issues and Opinions”, Dr. Gabor Racz (Ed.), ISBN: 978-953-307-813-7, InTech, and is hereby incorporated by reference in its entirety. One limitation of epidural needles in the prior art is that they do not have electrical insulation. Another limitation of epidural needles in the prior art is that they cannot functional as radiofrequency cannulae with a defined active tip.

Touhy needles with echogenic markings are well known. One example is the “Tuohy Ultrasonic” manufactured by Spectra Medical Devices of Wilmington, Mass., USA shown in the company's 2013 catalog, which is incorporated herein by reference in full. The tuohy needle distal end has a slight curve directly opposite the bevel. One limitation of echogenic tuohy needles in the prior art is that the shaft curvature is not configured for steering of the needle within tissue. Another limitation of echogenic tuohy needles in the prior art is that they do not have a bend in their shafts that is 5 mm or more from their most distal point. Another limitation of echogenic tuohy needles in the prior art is that they do not have electrical insulation along their shafts. Another limitation of echogenic tuohy needles in the prior art is that they are not configured for radiofrequency lesioning.

US Patent Applications 2012/009504 A1 by Massengale et al describes an echogenic nerve block apparatus. In FIG. 2D, Massengale shows a needle “body or shaft 24 that terminates in a generally flat, planar surface 26. In this particular example, the needle has a slight curve or bends 27 near the tip of the needle that defines that flat planar surface 26 . . . . The needle illustrated in FIG. 2D is sometimes referred to as a TUOHY needle or a needle having a TUOHY-type point.” One limitation of the art in Massengale is that the needle shaft is substantially straight. One limitation of the art in Massengale is that the slight curve in the needle is not 5 mm or more from the distal point of the needle. One limitation of the art in Massengale is that the needles cannot be rotated into a position that reduces the angle of incidence of incoming ultrasound waves over a substantial length of the needle, for example a length of 5 mm or more. One limitation of the art in Massengale is that the needles shown are not RF cannulae. Massengale also shows “soft tissue tunneling devices [that] include an elongate shaft having a rounded distal end. The distal end and/or the elongate shaft may be made echogenic in a manner similar to the echogenic needle and/or catheter as described above. These devices may further include a handle secured to the shaft in which the handle is configured to permit a user of the tunneling device to manually manipulate the tunneling device. The elongate shaft may be malleable so as to permit a shape of the shaft to be altered prior to use of the tunneling device. For example, the shaft may have a non-linear shape including, but not limited to, a curved shape.” One limitation of the soft tissue tunneling devices disclosed in Massengale is that they are not needles with sharp tips. One limitation of the soft tissue tunneling devices disclosed in Massengale is that they are not RF cannulae. One limitation of the soft tissue tunneling devices disclosed in Massengale is that they are not configured to delivery RF energy for therapeutic purposes.

Needles are used in medicine for a variety of applications, including without limitation injecting of anesthetics, neurolyltic agents, injection of medicine, and injection of radiographic contrast. Needles are used in medicine to inject and insert substances and devices in a variety of targets in the human body including muscles, nerves, organs, blood vessels, bone, connective tissue, body cavities, bodily spaces, bodily potential spaces.

U.S. Pat. No. 4,582,061 authored by F J Fry, in which a straight needle with ultrasonically reflective displacement scale is presented, is hereby incorporated by reference in its entirety. One limitation of this invention is that the echogenic probe has a straight shaft.

U.S. Pat. No. 4,869,259 authored by D J Elkins, in which an echogenically enhanced surgical instrument and method for production thereof is presented, is hereby incorporated by reference in its entirety. One limitation of this invention is that the echogenic needle has a straight shaft.

U.S. Pat. No. 5,081,991 authored by Bosley et al., in which echogenic devices material and method is presented, is hereby incorporated by reference in its entirety. One limitation of this invention is that the echogenic needle has a straight shaft.

U.S. Pat. No. 5,383,466 authored by L. Partika, in which an instrument having enhanced ultrasound visibility is presented, is hereby incorporated by reference in its entirety. One limitation of this invention is that the echogenic needle has a straight shaft.

U.S. Pat. No. 5,490,521 authored by R E Davis and G L McLellan, in which an ultrasound biopsy needle is presented, is hereby incorporated by reference in its entirety. One limitation of this invention is that the ultrasound needle has a straight shaft.

U.S. Pat. No. 5,759,154 authored by D V Hoyns, in which a print mask technique for echogenic enhancement of medical device is presented, is hereby incorporated by reference in its entirety. One limitation of this invention is that the echogenic needle has a straight shaft.

U.S. Pat. No. 5,921,933 authored by R G Sarkins et al., in which medical devices with echogenic coatings are presented, is hereby incorporated by reference in its entirety. One limitation of this invention is that the echogenic needle has a straight shaft.

US Patent Application 2009/0137906 A1 authored by Maruyama et al., in which an ultrasound piercing needle is presented, is hereby incorporated by reference in its entirety. One limitation of this invention is that the echogenic needle has a straight shaft. Another limitation is that the needle is not a radiofrequency cannula. Another limitation is that the needle is not a radiofrequency electrode. Another limitation is that the needle is not a microwave antenna. Another limitation is that the means of echogenic enhancement does not utilize both macroscopic depressions in the needle surface and microscopic roughing of the needle surface.

The present invention seeks to overcome the limitations and disadvantages of the prior art.

### SUMMARY OF THE INVENTION

The present invention relates generally to the application of echogenic markers to radiofrequency cannulae and electrodes. An advantage of the present invention is that radiofrequency probes can be more easily visualized and directed in the human body by means of ultrasound guidance.

The present invention relates generally to the application of echogenic markers and a curved tip to a medical needle, including radiofrequency cannulae, injection needles, biopsy needles, microwave antennae, and spinal needles. An advantage of the present invention is that medical needles can be more easily visualized and directed in the human body by means of ultrasound guidance when the needle is inserted at a steep angle relative to the ultrasound beam.

The present invention relates generally to the application of echogenic markers to medical probes wherein multiple types of echogenic markers are applied to the same probe and the multiple types of echogenic markers have different spatial scale and angles. An advantage of the present invention is that medical needles can be more easily visualized and directed in the human body by means of ultrasound guidance for a wide range of probe insertion angles relative to the ultrasound transceiver.

In one aspect, a radiofrequency probe can have an echogenic feature.

In certain embodiments, the probe can have a curved tip. The probe can be a cannula, an electrode, or a unitized injection electrode. The probe can be tissue-piercing. The probe can have a stiff shaft. The probe can include a shaft is composed of metal. The probe can be a radiofrequency cannula with a bevel configured for placement in the epidural space. The probe can be a needle configured to introduce a catheter. The probe can have a distal and proximal end, and a first and a second indentation in a surface of the probe, wherein the first indentation includes a distal aspect having a first angle relative to the surface of the probe, and the second indentation includes a distal aspect having a second angle relative to the surface of the probe.

In another aspect, a needle can have a curved tip and an echogenic feature.

In certain embodiments, the needle includes a shaft is composed of metal. The needle can be a radiofrequency cannula, part of a unitized radiofrequency electrode, an epidural needle, or a spinal needle. The needle can be configured for effecting a nerve block. The needle can have a distal and proximal end, and a first and a second indentation in a surface of the needle, wherein the first indentation includes a distal aspect having a first angle relative to the surface of the needle, and the second indentation includes a distal aspect having a second angle relative to the surface of the needle.

In another aspect, a medical probe can have a first echogenic feature and a second echogenic feature, wherein the first echogenic feature is an indentation in the surface of the probe, and the second echogenic feature is a roughing of the surface of the probe. The first and second feature can be in the same location on the shaft.

In certain embodiments, the probe can be a needle, a radiofrequency cannula, a radiofrequency electrode, an internally-cooled radiofrequency electrode, a radiofrequency needle, an epidural needle, a biopsy needle, or a spinal needle. The probed can have a curved tip, a sharp bevel, or a blunt tip.

In certain embodiments, the roughing of the probe's surface can be produced by sandblasting or beadblasting.

In certain embodiments, the indentation can have a three-sided pyramidal shape.

In certain embodiments, the probe can include a shaft having a multitude of echogenic indentations.

In another aspect, a radiofrequency cannula can have at least one echogenic feature.

In another aspect, a curved-tip radiofrequency cannula can have at least one echogenic feature.

In another aspect, a radiofrequency electrode can have at least one echogenic feature.

In another aspect, a curved medical needle can have at least one echogenic feature.

### DESCRIPTION OF THE DRAWINGS

FIG. 1 is an illustration of an exemplary insulated, curved echogenic needle and its associated stylet and electrode.

FIG. 2 is an illustration of an exemplary insulated, straight echogenic needle and its associated stylet and electrode.

FIG. 3 is an illustration of an exemplary insulated, curved, echogenic, unitized electrode.

FIG. 4 is an illustration of an exemplary curved, echogenic needle.

FIG. 5 is an illustration of an exemplary echogenic marker.

FIGS. 6A-C are illustrations of an exemplary echogenic marker.

FIGS. 7A-F are illustrations is an illustration of exemplary echogenic markers in a cross-sectional view.

FIG. 8A is an illustration of exemplary straight, electrically-insulated needle each placed in tissue and visualized using an ultrasound beam.

FIG. 8B is an illustration of exemplary curved, electrically-insulated needle each placed in tissue and visualized using an ultrasound beam.

FIG. 9A is an illustration of an exemplary echogenic marker on a straight, electrically-insulated needle each placed in tissue and visualized using an ultrasound beam.

FIG. 9B is an illustration of an exemplary echogenic marker on a curved, electrically-insulated needle each placed in tissue and visualized using an ultrasound beam.

### DETAILED DESCRIPTION

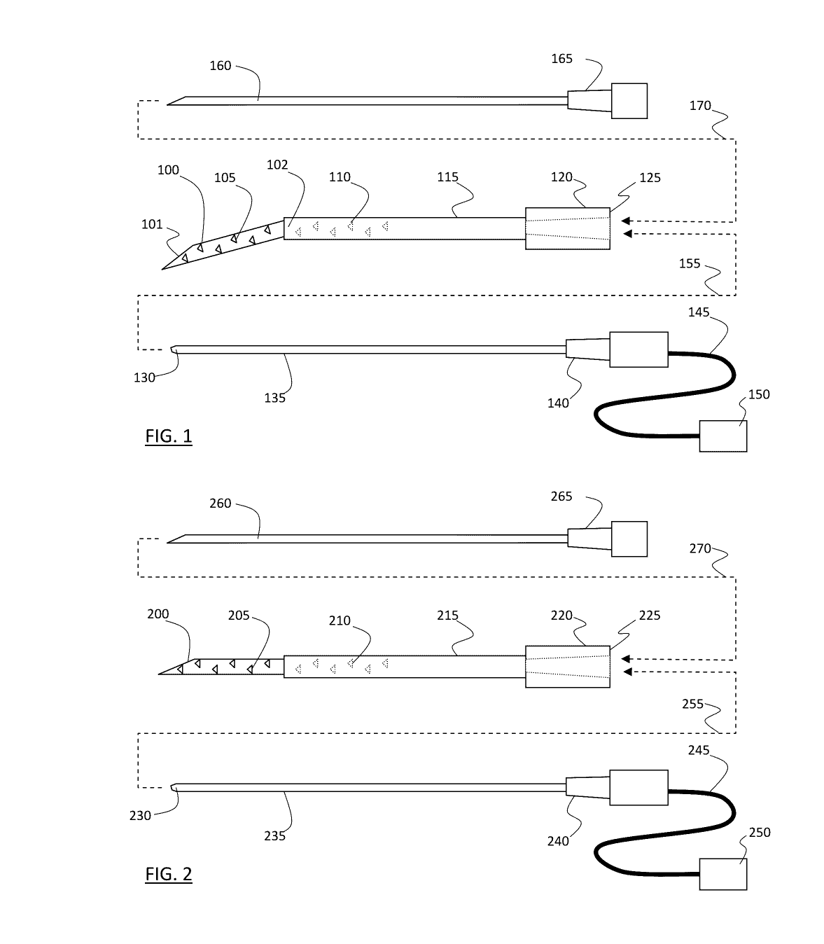

Referring to FIG. 1, a needle with shaft 100 is shown. The shaft 100 be substantially cylindrical. The needle can be hollow with an inner lumen. The inner lumen of the shaft 100 can open to the outside via a hole in the tip of the shaft 100 or holes along the shaft. The needle has a sharpened distal end, and is terminated by hub 120 at its proximal end. The needle can configured to penetrate biological tissue, such as the skin's surface, soft tissue around the spine, visceral organs, limbs, muscles, blood vessels, the liver, the kidney, the prostate, and other human and animal tissues. The needle's distal end can have a bevel 101. The needle can be a biopsy needle. The needle's distal end 101 can have a tissue-piercing geometry, such as a chiba tip. The needle's distal end 101 can have a rounded tip and stiff shaft capable of piecing tissue. The needle's distal end 101 can have an epidural geometry, such as a tuohy tip. The needle can be radiofrequency cannula. The needle can be configured to deliver high frequency electrical energy to tissue. The needle can be configured for radiofrequency lesioning. The needle can be configured for pulsed radiofrequency treatment. The needle can be configured for lesioning of nervous tissue. The needle can be configured for lesioning of cancerous tissue. The needle can be configured for insertion into blood vessels. The needle can be configured by insertion in the epidural space. The needle can be configured for use in and around the spine. The needle can be configured for a nerve block procedure. The shaft can be composed of a metallic substance such as stainless steel. The shaft 100 can be rigid. The shaft 100 can be composed of an electrically conductive substance. The metallic shaft 100 is covered with electrical insulation 115. The electrical insulation 115 can be configured to transmit sound waves without substantially impeding or scattering them. The electrical insulation 115 can be a plastic coating. The needle's active tip is the metallic portion of the shaft which is not covered with insulation 115, ie the region of the shaft that is distal to the insulation. The needle's hub 120 can be a luer hub. The needle's hub 120 can a locking luer hub. The needle's hub 120 can admit a syringe or tubing for injection of fluids, such as saline, steroids, anesthetics, neurolytic agents, coagulants, chemotherapy agents, and other medical fluids.

The shaft 100 can be bent at its distal end. The angle of the bend can be 5 degrees. The angle of the bend can be 10 degrees. The angle of the bend can be 15 degrees. The angle of the bend can be 20 degrees. The angle of the bend can be 25 degrees. The angle of the bend can be 30 degrees. The angle of the bend can be a value greater than 30 degrees. The angle of the bend can be a value less than 30 degrees. The shaft 100 can be straight.

The bend 102 in the shaft can be positioned substantially at the same location as the distal end of the electrical insulation 115. The bend 102 in the shaft can be positioned proximal to the distal end of the electrical insulation 115. The bend 102 in the shaft can be positioned distal to the distal end of the electrical insulation 115. The bend 102 in the shaft 100 can be a curve that starts at a proximal point along the shaft, and continues all the way to the most distal point of the shaft 100. The bend 102 in the shaft 100 can be a curve that starts and stops proximal to the most distal point of the shaft. The bend 102 in the shaft 100 can have lengths of straight shaft both distal and proximal to the shaft, as illustrated in FIG. 1. The bend 102 can be 1 mm from the most distal point of the shaft 100. The bend 102 can be 2 mm from the most distal point of the shaft 100. The bend 102 can be 3 mm from the most distal point of the shaft 100. The bend 102 can be 4 mm from the most distal point of the shaft 100. The bend 102 can be 5 mm from the most distal point of the shaft 100. The bend 102 can be 6 mm from the most distal point of the shaft 100. The bend 102 can be 7 mm from the most distal point of the shaft 100. The bend 102 can be 8 mm from the most distal point of the shaft 100. The bend 102 can be 9 mm from the most distal point of the shaft 100. The bend 102 can be 10 mm from the most distal point of the shaft 100. The bend 102 can be more than 10 mm from the most distal point of the shaft 100. The bend 102 can be between 5 mm and 10 mm from the most distal point of the shaft. The bend 102 can be configured to improve the steerability of the shaft 100 through tissue.

The echogenic markers 105 can be positioned on the active tip of the shaft 100, and the echogenic markers 110 can be positioned under or within the insulation 115. The echogenic markers 105 can be positioned distal to the bent section of the shaft 100, and the echogenic markers 110 can be positioned proximal to the bent section of the shaft 100. The echogenic markers 105 can be positioned along the bent section of the shaft 100, and the echogenic markers 110 can be positioned proximal to the bent section of the shaft 100. The cluster of markers 105 can appear different to the cluster of markers 110 when viewed using ultrasound imaging. The cluster of markers 105 can be physically separated from the cluster of markers 110 so that the two clusters can be distinguished when viewed using ultrasound imaging. In one embodiment, the echogenic markers 105 can be omitted. In one embodiment, the echogenic markers 110 can be omitted.

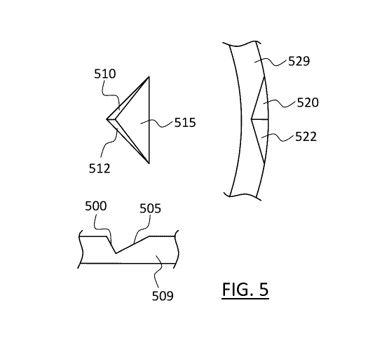

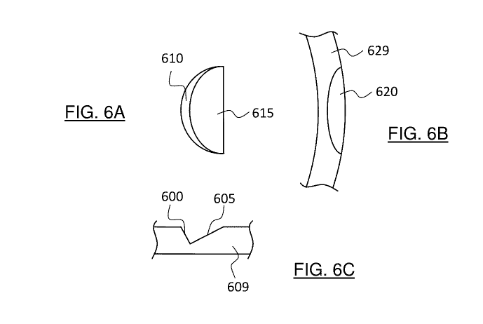

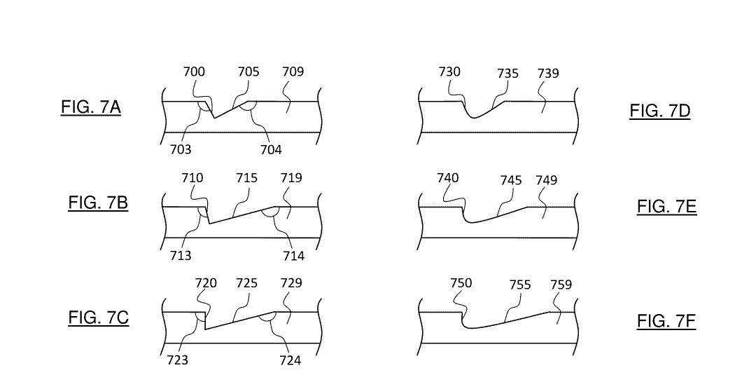

The echogenic markers 105 and 110 can be configured to enhance the needle's shaft visibility when viewed with ultrasound imaging. For example, the echogenic markers 105 and 110 can be configured such that when the needle is inserted to a living body and an ultrasound transceiver in contact with the skin of the living body is directed at the needle, the ultrasound image of the needle is enhanced relative to what its image if the needle shaft did not have the echogenic markers 105 and 110. The echogenic markers 105 and 110 can be indentations in the surface of the shaft 100. The echogenic markers 105 and 110 can be produced by means of stamping a shape or shapes into the shaft 100. The echogenic markers 105 can be produced by means of sand blasting the shaft 100. The echogenic markers 105 and 110 can be produced by means of bead blasting the shaft 100. The echogenic markers 105 can be produced by means of roughing the surface of the shaft 100. The echogenic markers 105 and 110 can be produced by means of laser ablation the surface of the shaft 100. The echogenic markers 105 and 110 can be linear depressions in the surface of the shaft 100. The echogenic markers 105 and 110 can be circumferential grooves in the surface of the shaft 100. The echogenic markers 105 and 110 can be material variations in the insulation 115. The echogenic markers 105 can produce echogenic enhancement by a different means than the echogenic markers 110. The echogenic markers 105 and 110 can each be a multitude of markers, each of which markers have a size in the range 0.005 and 0.020 inches on the surface of the needle shaft 100, and depth between 0.002 and 0.005 inches into the surface of the needle shaft 100. The echogenic markers 105 and 110 can include both macroscopic echogenic dents (examples of one of which include the markers shown in FIG. 5, FIGS. 6A-C, and FIGS. 7A-F) in the surface of shaft 100 and a microscopic roughing of the surface (such as that produced by sandblasting or beadblasting) of the shaft 100; one advantage of this embodiment is that the macroscopic dents can reflect ultrasound waves when the shaft 100 is positioned at a steep angle relative to the ultrasound transceiver and the microscopic surface roughing produces an enhanced image of the entire shaft when the shaft 100 is positioned at shallow angles relative to the ultrasound transceiver. In one example, the echogenic marker 105 can be produced by sandblasting the surface of the shaft 100 and then producing at least one macroscopic dent in the surface of the shaft 100 where the sandblasting was applied. In one example, the echogenic marker 105 can be produced by producing at least one macroscopic dent in the surface of the shaft 100 and then sandblasting the surface of the shaft 100 at and around the location or locations of the said at least one macroscopic indentation. In one example, the echogenic marker 105 can be a macroscopic indentation at a first location on the shaft 100 and sandblasting at a second location on the shaft 100.

The needle's inner lumen can admit a stylet 160 with cap 165. The stylet cap 165 can engage with the needle hub 120. The stylet can fill some or all of the needle's hollow shaft to facilitate insertion of the needle into biological tissue. The stylet's shaft 160 can be composed of stainless steel. The stylet's shaft 160 can be composed of a plastic. The stylet's shaft 160 can be substantially rigid. The stylet's shaft 160 can be substantially flexible. When the stylet's cap 165 is fully engaged with the needle's hub 120, the stylet's distal end can be substantially flush with the distal end of the needle shaft 100. When the stylet's cap 165 is fully engaged with the needle's hub 120, the stylet's 160 distal end can extend beyond the distal end of the needle shaft. The stylet 160 can be a flexible material, and when the stylet's cap 165 is fully engaged with the needle's hub 120, the stylet's 160 distal end can extend beyond the distal end of the needle shaft to provide tactile feedback that an structure, such as the dura matter, has been encountered as the needle is advanced into bodily tissue without piercing that structure.

The needle's inner lumen can admit an electrode with distal tip 130, shaft 135, hub 140, cable 145, and connector 150. The electrode 135 can be a radiofrequency electrode, well known to one skilled in the art. The electrode hub 140 can engage with the cannula hub 120. The electrode tip 130 can house a temperature sensor. The connector 150 can couple the electrode to an electrical power supply, such as a nerve stimulator, radiofrequency generator, or PENS generator. The electrode 135 can be an internally-cooled electrode, such as by fluid circulating within the electrode shaft.

In one embodiment, the cannula hub 120 can have an additional connection so that fluid can be injected at the same time the electrode 135 is fully inserted into the cannula shaft 100 and the electrode hub 140 is fully engaged into the cannula hub 120. In another embodiment, the electrode hub 140 has an additional fluid connection so that fluid can be injected into and through the cannula shaft 100 at the same time the electrode 135 is fully inserted into the cannula shaft 100 and the electrode hub 140 is fully engaged into the cannula hub 120.

The active tip of the cannula shaft 100 can be less than 1 mm in length. The active tip of the cannula shaft 100 can be 1 mm in length. The active tip of the cannula shaft 100 can be 2 mm in length. The active tip of the cannula shaft 100 can be 4 mm in length. The active tip of the cannula shaft 100 can be 5 mm in length. The active tip of the cannula shaft 100 can be 6 mm in length. The active tip of the cannula shaft 100 can be 10 mm in length. The active tip of the cannula shaft 100 can be 15 mm in length. The active tip of the cannula shaft 100 can be 20 mm in length. The active tip of the cannula shaft 100 can be 30 mm in length. The active tip of the cannula shaft 100 can be 40 mm in length. The active tip of the cannula shaft 100 can be 50 mm in length. The active tip of the cannula shaft 100 can be 60 mm in length. The active tip of the cannula shaft 100 can be greater than 60 mm in length. The active tip of the cannula can be between 1 mm and 60 mm in length.

The cannula shaft's diameter can be less than 23 gauge. The cannula shaft's diameter can be 22 gauge. The cannula shaft's diameter can be 21 gauge. The cannula shaft's diameter can be 20 gauge. The cannula shaft's diameter can be 18 gauge. The cannula shaft's diameter can be 16 gauge. The cannula shaft's diameter can be 15 gauge. The cannula shaft's diameter can be 14 gauge. The cannula shaft's diameter can be greater than 16 gauge. The cannula shaft's diameter can be between 23 and 14 gauge.

The cannula shaft's length can be less than 5 cm. The cannula shaft's length can be 5 cm. The cannula shaft's length can be 10 cm. The cannula shaft's length can be 15 cm. The cannula shaft's length can be 20 cm. The cannula shaft's length can be 25 cm. The cannula shaft's length can be less than 5 cm. The cannula shaft's length can be between 5 cm and 25 cm. The cannula shaft's length can be greater than 25 cm.

The cannula shaft's diameter can be less than 23 gauge. The cannula shaft's diameter can be 22 gauge. The cannula shaft's diameter can be 21 gauge. The cannula shaft's diameter can be 20 gauge. The cannula shaft's diameter can be 18 gauge. The cannula shaft's diameter can be 16 gauge. The cannula shaft's diameter can be greater than 16 gauge. The cannula shaft's diameter can be between 23 and 16 gauge.

In one embodiment, the needle does not admit a stylet 160.

In one embodiment, a radiofrequency cannula has both a bent distal tip and markers that enhance said radiofrequency cannula's image when said cannula is positioned in the human body and viewed with an ultrasound imaging apparatus. One advantage of this embodiment is that a radiofrequency cannula can be easily positioned using ultrasound guidance. One advantage of this embodiment is that a radiofrequency cannula can be easily positioned near soft tissue anatomy that is visible using ultrasound imaging. One advantage of this embodiment is that a radiofrequency cannula can be easily positioned near soft tissue anatomy that is visible using ultrasound imaging and not visible using radiographic imaging, such as x-ray. One advantage of this embodiment is that a curved radiofrequency cannula can be steered by a physician during its placement in bodily tissue. One advantage of this embodiment is that a curved tip can be used to make the tip more perpendicular to the ultrasound transceiver than is the shaft. One advantage of this embodiment is that a curved tip can be used to make the tip more perpendicular to an ultrasound transceiver than is the shaft, and thus allow both an enhanced ultrasound image of the tip and a steep approach to target anatomy.

It is understood that in other embodiments electrical insulation can be applied in multiple segments to the cannula shaft 100, including the placement of insulation distal to the active tip. It is understood that the cannula shaft 100 can have an overall curved shape. It is understood that the cannula shaft 100 can have multiple curves.

In another embodiment, the device in FIG. 1 can have a substantially straight shaft. In another embodiment, the angle 102 can be zero.

Referring to FIG. 2, another embodiment of the present invention is shown in which the insulated cannula has a straight shaft 200. The elements presented in FIG. 2 and analogous to those presented in FIG. 1. In one embodiment of the present invention, a radiofrequency cannula has a straight shaft 200 and markers that enhance said radiofrequency cannula's image when said cannula is positioned in the human body and viewed with an ultrasound imaging apparatus. One advantage of this embodiment is that a radiofrequency cannula can be easily positioned near soft tissue anatomy that is visible using ultrasound imaging. One advantage of this embodiment is that a radiofrequency cannula can be easily positioned near soft tissue anatomy that is visible using ultrasound imaging and not visible using radiographic imaging, such as x-ray.

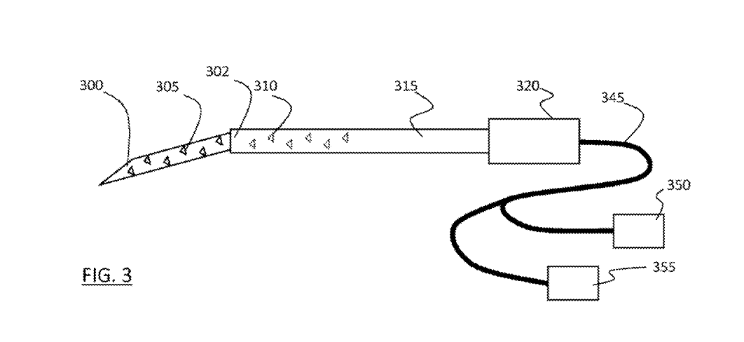

Referring to FIG. 3, an electrode is presented wherein the electrode's shaft 300 has a bent distal end, the electrode has a hub 320 at its proximal end, the electrode's shaft is covered by electrical insulation 315 along its proximal length which forms an uninsulated active tip near or at the electrode's distal end, the electrode's bent distal active tip has one or more echogenic markers 305, the electrode's shaft can have additional echogenic markers 310, the cabling 345 connects to an electrical connector 350 for connection to a radiofrequency generator or nerve stimulator and delivery of radiofrequency energy or stimulation waveforms to the active tip of the electrode, and the cabling 345 connects to a fluid connector 355 for delivery of fluid through the electrode's hollow shaft 300 and out from holes along the shaft 300 or at the distal tip of the shaft 300. The echogenic markers 305 and 310 can be configured to be visually distinguished when viewed using ultrasound imaging. In one embodiment, the shaft 300 can be straight over its entire length. In one embodiment, the electrode's shaft can be configured to pierce tissue. In one embodiment, the electrode's shaft can be sharpened. In one embodiment, the electrode is a radiofrequency electrode. In one embodiment, the electrode has a temperature sensor at its tip and is configured so that a radiofrequency generator can contain the measured temperature when radiofrequency power is delivered via the electrode into living tissue, such as that of a human body. In one embodiment, the electrode is a injection needle configured for stimulation-guided injections near or in nervous tissue. The dimensions of the active tip, the shaft length, the bend, and the shaft diameter can fall in the same ranges as those of the needle presented in FIG. 1. In one embodiment, the electrode omits the markers 310. In another embodiment, the apparatus 300, 302, 305, 310, 315, 320, 345, 350, 355 can be a microwave antenna, such as that used for medical tissue ablation. In another embodiment, the apparatus 300, 302, 305, 310, 315, 320, 345, 350, 355 can be a probe for use in biological tissue, such as the human body. In another embodiment, the device in FIG. 3 can be a unitized injection electrode, such as the electrodes shown U.S. Pat. No. 7,862,563 by Cosman et al.

In another embodiment, the electrode presented in FIG. 3 can have a substantially straight shaft. In another embodiment, the angle of bend 302 can be zero.

It is understood that the probe presented in FIG. 3 can have multiple electrical contact (as in a bipolar electrode), multiple segments of insulation, and multiple curves.

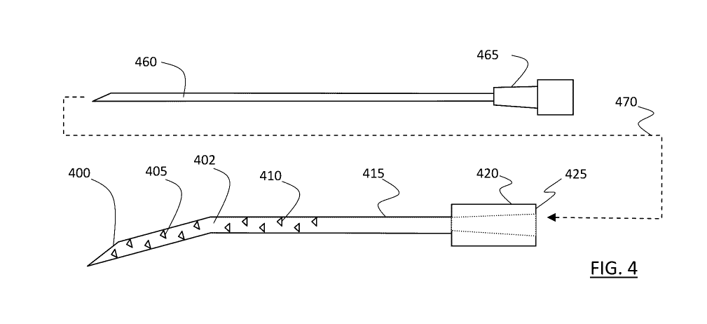

Referring to FIG. 4, a needle 400 is presented that has a bent tip and echogenic markers, and that does not have electrical insulation. The needle can be hollow for injection of fluid and the introduction of the stylet 460. The needle's shaft 415 is not insulated. The needle 400 has echogenic elements 405 and 410. The needle's shaft can be rigid. The shaft of the needle 400 can be metallic, such as a stainless steel hypotube. The needle 400 can be tissue-piecing. The needle 400 can have a sharpened tip. The elements of the needle presented in FIG. 4 are analogous to those of the needle presented in FIG. 1. In one embodiment, the needle 400 is a spinal needle. In one embodiment, the needle 400 is for injection in or near nervous tissue. In one embodiment, the needle 400 is for injection in the epidural space. In one embodiment, the needle 400 is for injection in blood vessels. One advantage of the needle 400 is that the echogenic markers can enhance the image of the needle 400 when placed in the human body and viewed using an ultrasound probe placed at the skin's surface. One advantage of the needle 400 with a curved tip is that the needle can be rotated so that the tip is more perpendicular to the ultrasound wavefront without changing the trajectory of the needle's shaft 415. One advantage of the needle 400 with a curved tip is that the needle can be rotated so that the tip is more perpendicular to the ultrasound wavefront without changing the trajectory of the needle's shaft 415, and thereby the ultrasound image of the needle's tip can be enhanced even when the needle's shaft 415 is substantially parallel to the ultrasound waves. In one embodiment, the needle 400 does not have a bent tip. In one embodiment, the needle 400 has a shaft that is straight over its entire length.

The bend 402 in the shaft of needle 400 can be a curve that starts at a proximal point along the shaft, and continues all the way to the most distal point of the shaft 400. The bend 402 in the shaft 400 can be a curve that starts and stops proximal to the most distal point of the shaft. The bend 402 in the shaft 400 can have lengths of straight shaft both distal and proximal to the shaft, as illustrated in FIG. 1. The bend 402 can be 1 mm from the most distal point of the shaft 400. The bend 402 can be 2 mm from the most distal point of the shaft 400. The bend 402 can be 3 mm from the most distal point of the shaft 400. The bend 402 can be 4 mm from the most distal point of the shaft 400. The bend 402 can be 5 mm from the most distal point of the shaft 400. The bend 402 can be 6 mm from the most distal point of the shaft 400. The bend 402 can be 7 mm from the most distal point of the shaft 400. The bend 402 can be 8 mm from the most distal point of the shaft 400. The bend 402 can be 9 mm from the most distal point of the shaft 400. The bend 402 can be 10 mm from the most distal point of the shaft 400. The bend 402 can be more than 10 mm from the most distal point of the shaft 400. The bend 402 can be between 5 mm and 10 mm from the most distal point of the shaft. The bend 402 can be configured to improve the steerability of the shaft 400 through tissue.

Referring to FIG. 5, presented in three perpendicular views is one example of an echogenic marker in the shaft of a needle, electrode, or probe, such as those presented in FIGS. 1, 2, 3, and 4. The marker is depression in the side of the probe, and can be formed, for example, by cutting, laser ablating, stamping, or pressing into the side of the probe. Elements 510, 512, 515 present a view of the echogenic marker's incut planes looking in the radial direction from the outside of the probe, ie as the marker appears looking at the probe's shaft from the outside. The length and the width of the echogenic marker can each be in the range 0.005 inches to 0.020 inches. The length and the width of the echogenic marker can each be less than 0.005 inches. The length and the width of the echogenic marker can each be greater than 0.020 inches. Elements 500, 505, 509 present a cross-sectional view of the said echogenic marker though the probe's wall 509, of which only a short segment is shown, in the radial-axial direction. Element 500 and 505 represent surfaces that are on the more outer surface of the probe's wall 509; the bottom of wall 509 is inside the inner lumen of the probe's shaft. Element 500 is a cross section through the intersection of planes 510 and 512. Element 505 shows a cross-sectional cut of plane 515. Elements 520, 522, 529 present a view of the said echogenic marker constructed by cutting through the probe's wall 529, of which only a segment is shown, perpendicular the axis of the cylindrical probe, and looking in the direction of planes 520 and 522, which correspond to planes 510 and 512, respectively, in the view 510, 512, 515. Element 500 is a cross section through the intersection of planes 520 and 522.

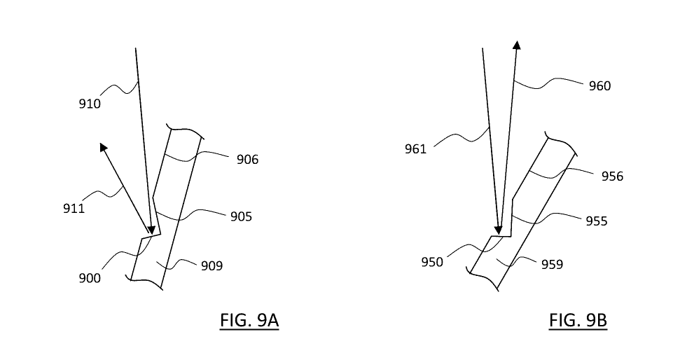

The marker in FIG. 5 can be oriented with the long axis of the probe's shaft; for example, the planes 510 and 512 can be distal to the plane 515. The probe's wall 509, 529 can the wall of a stainless steel tube. For example, for a shaft that is 21 gauge tubing with outer diameter 0.032 inches and inner diameter 0.020, the thickness of wall 509, 529 is 0.006 inches. The depth of the marker in the wall 509, 529 can be less than the thickness of the wall. The depth of the marker in the wall 509, 529 can be less than 0.002 inches. The depth of the marker in the wall 509, 529 can be 0.002 inches. The depth of the marker in the wall 509, 529 can be 0.003 inches. The depth of the marker in the wall 509, 529 can be 0.004 inches. The depth of the marker in the wall 509, 529 can be 0.005 inches. The depth of the marker in the wall 509, 529 can be 0.006 inches. The depth of the marker in the wall 509, 529 can be greater than 0.006 inches. The depth of the marker in the wall 509, 529 can be in the range 0.002 to 0.006 inches. The depth of the marker in the wall 509, 529 can be equal or greater to the wall thickness so that the marker provides outlets for fluid outflow from the inner lumen of the shaft. The three planes 510, 512, and 515 can be mutually orthogonal to each other. The three planes 510, 512, 515 can be non-perpendicular to each other. Planes 510 and 512 can be perpendicular to each other, and plane 515 can be non-perpendicular to plane 510 and non-perpendicular to plane 512. The marker in FIG. 5 can be constructed so that plane 515 has a more shallow angle with respect to the outside of the probe than do planes 510 and 512; in this embodiment, line 505 is closer to parallel with the outer wall of the probe shaft 509 than is line 500; in this embodiment, when the planes 510 and 512 are positioned distal to plane 515 and the probe is placed in a living body within the fan of an ultrasound probe, the shallow angle of 515 occludes less of planes 510 and 512 from ultrasound beam and planes 510 and 512 are more perpendicular to the ultrasound beam (as shown, for example, in FIG. 9A). In one embodiment, multiple instances of the marker shown in FIG. 5 can be placed at multiple position on the shaft of a probe like those shown in FIGS. 1, 2, 3, and 4; one advantage of using multiple markers is to improve the signal to noise ratio of the probe's signal in an ultrasound image; another advantage of using multiple markers to the enhance the probe's image when viewed from different angles using ultrasound imaging. In one embodiment, multiple instances of the marker shown in FIG. 5 are placed at specific locations which can be used to judge scale and/or distinguish parts of the probe (such the tip) in an ultrasound image.

Referring to FIGS. 6A-C, presented in three perpendicular views is one example of an echogenic marker in the shaft of a needle, electrode, or probe, such as those presented in FIGS. 1, 2, 3, and 4. The marker is depression in the side of the probe, and can be formed, for example, by cutting, laser ablating, stamping, or pressing into the side of the probe. Elements 610 and 615 present a view of the echogenic marker's incut surfaces looking in the radial direction from the outside of the probe, ie as the marker appears looking at the probe's shaft from the outside. The surface 610 can be curved. The surface 615 can be curved. The surface 615 can be planar. Elements 600, 605, 609 present a cross-sectional view of the said echogenic marker though the probe's wall 609 in the radial-axial direction. Element 600 and 605 represent surfaces that are on the more outer surface of the probe's wall 609, of which only a short segment is shown; the bottom of wall 609 is inside the inner lumen of the probe's shaft. Element 600 is a cross section through surface 610. Element 605 shows a cross-sectional cut of surface 615. Elements 620 and 629 present a view of the said echogenic marker constructed by cutting through the probe's wall 629, of which only a segment is shown, perpendicular the long axis of the cylindrical probe, and looking in the direction of surface 620, which corresponds to plane 610 in the view 610, 615. Element 600 is a cross section through the surface 620.

The marker can be oriented with the long axis of the probe's shaft; for example, the surface 610 can be distal to the surface 615. The probe's wall 609, 629 can the wall of a stainless steel tube. For example, for a shaft that is 21 gauge tubing with outer diameter 0.032 inches and inner diameter 0.020, the thickness of wall 609, 629 is 0.006 inches. The depth of the marker in the wall 609, 629 can be less than the thickness of the wall. The depth of the marker in the wall 609, 629 can be less than 0.002 inches. The depth of the marker in the wall 609, 629 can be 0.002 inches. The depth of the marker in the wall 609, 629 can be 0.003 inches. The depth of the marker in the wall 609, 629 can be 0.004 inches. The depth of the marker in the wall 609, 629 can be 0.005 inches. The depth of the marker in the wall 609, 629 can be 0.006 inches. The depth of the marker in the wall 609, 629 can be greater than 0.006 inches. The depth of the marker in the wall 609, 629 can be in the range 0.002 to 0.006 inches. The depth of the marker in the wall 609, 629 can be equal or greater to the wall thickness so that the marker provides outlets for fluid outflow from the inner lumen of the shaft. The marker in FIG. 5 can be constructed so that plane 615 has a more shallow angle with respect to the outside of the probe than does surface 610; in this embodiment, line 605 is closer to parallel with the outer wall of the probe shaft 609 than is line 600; in this embodiment, when the surface 610 is positioned distal to surface 615 and the probe is placed in a living body within the fan of an ultrasound probe, the shallow angle of 615 occludes less of surface 610 from ultrasound beam and surface 610 is more perpendicular to the ultrasound beam (as shown, for example, in FIG. 9A). In one embodiment, multiple instances of the marker shown in FIGS. 6A-C can be placed at multiple position on the shaft of a probe like those shown in FIGS. 1, 2, 3, and 4; one advantage of using multiple markers is to improve the signal to noise ratio of the probe's signal in an ultrasound image; another advantage of using multiple markers to the enhance the probe's image when viewed from different angles using ultrasound imaging. In one embodiment, multiple instances of the marker shown in FIGS. 6A-C are placed at specific locations which can be used to judge scale and/or distinguish parts of the probe (such the tip) in an ultrasound image. In one embodiment, a single probe such as one of those presented in FIGS. 1, 2, 3, and 4, contain multiple type of dent-like markers, for example, both markers of the type presented in FIG. 5 and markers of the type presented in FIGS. 6A-C; one advantage of this embodiment is that it can improve visibility of the probe under different conditions.

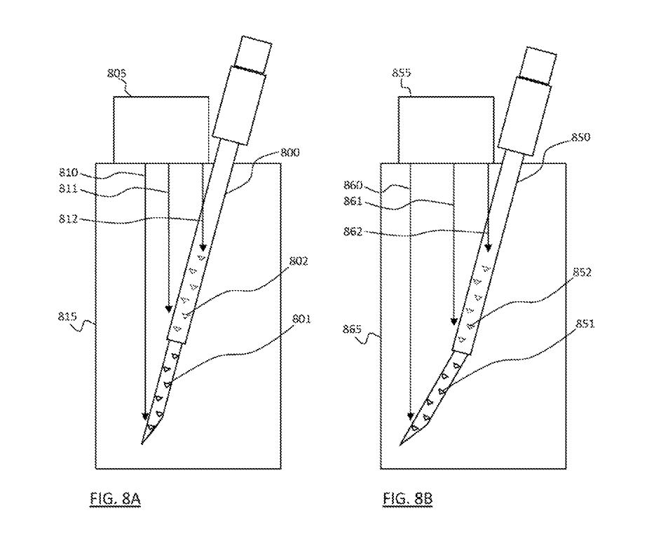

Referring to FIGS. 7A-F, presented in cross-section are six embodiments of individual echogenic markers that can be incorporated into a probe like those presented in FIGS. 1, 2, 3, and 4. Each marker is shown in an axial-radial cross-sectional view similar to that of marker 500, 505, and 509 of FIGS. 6A-C and that of marker 600, 605, and 609 of FIGS. 6A-C. For each example marker, elements further to the left are more distal along the probe's shaft (ie closer to the tissue-penetrating end of the probe), and element further to the right are more proximal along the probe's shaft (ie closer to the hub of the probe). For the marker shown by surface 700, surface 705, and wall 709, angle 703 is the angle between surface 700 and the outer surface of the shaft, and angle 704 is the angle between surface 705 and the outer surface of the shaft. The angle 703 can be small than the angle 704; one advantage of this embodiment is that when the shaft is viewed at a steep angle relative to the ultrasound probe (as shown, for example, in FIG. 8A and FIG. 8B), the shallow angle of surface 705 relative to the probes surface allows ultrasound pulses to bounce off surface 700. For the marker shown by surface 710, surface 715, and wall 719, angle 713 is the angle between surface 710 and the outer surface of the shaft, and angle 714 is the angle between surface 715 and the outer surface of the shaft. The angle 713 is smaller than angle 703; as such, when the probe is placed at a steeper angle relative to the ultrasound beam, surface 710 is more perpendicular to the ultrasound beam and reflects more ultrasound waves back to the ultrasound probe, thereby increasing the ultrasound signal induced by the marker 710, 715, 719 relative to marker 700, 705, 709 at that angle. The angle 714 is larger than angle 704; as such, even at steep angles, surface 715 allows more ultrasound waves to contact surface 710 than it would if angle 714 had the same value as 704. The marker shown by surface 720, 725, and 729 is characterized by angle 723 that is closer to a right angle than are angles 703 and 713; as such, sound reflections back to the ultrasound transceiver are increased at very steep shaft angles. The marker shown by surface 720, 725, and 729 is characterized by angle 724 that is closer to 180 than are angles 704 and 714; as such, sound waves from the ultrasound transceiver are allowed an unimpeded path to surface 720 over a wider range of shaft angles than would be allowed were angle 724 equal in value to 704 or 714. The echogenic marker shown by shaft wall 739 and curved surface with distal part 730 and proximal part 735 is a curved depression in the surface of the shaft. One advantage of a curved, concave marker is that sound waves from the ultrasound transceiver can reflect off the surface and back toward the transceiver for a wide variety of shaft orientations relative to the transceiver. The distal part of the surface 730 can have a sharper curvature than the proximal part of the surface 735, so the proximal part does not block incoming ultrasound waves incident on the shaft at shallow angles and the distal part has a part roughly perpendicular to incoming sound waves incident on the shaft at shallow angles which can reflect said ultrasound waves back toward the ultrasound transceiver. The echogenic marker shown by shaft wall 749 and curved surface with distal part 740 and proximal part 745 is a curved depression in the surface of the shaft with a longer length in the axial direction (equivalent to the shaft's distal-proximal direction) than that of marker 730, 735, 739, and with a proximal part 745 that has a more gradual slope than the proximal part 735 of the marker. The shallower slope of proximal part 745 relative to proximal part 735 allows incoming sound waves to contact distal part 740 for steeper shaft angles relative to the ultrasound beam. The echogenic marker shown by shaft wall 759 and curved surface with distal part 750 and proximal part 755 is a curved depression in the surface of the shaft with a longer length in the axial direction (equivalent to the shaft's distal-proximal direction) than that of marker 740, 745, 749, and with a proximal part 755 that has a more gradual slope than the proximal part 745 of the marker. The shallower slope of proximal part 755 relative to proximal part 745 allows incoming sound waves to contact distal part 750 for steeper shaft angles relative to the ultrasound beam. The proximal part 750 can has generally steeper curvature than proximal part 740; as such, when this marker is used on a probe that is inserted more parallel to the central axis of the ultrasound beam, the proximal part 750 will be more likely to reflect ultrasound signals back toward the ultrasound transceiver.

In one embodiment, a single probe such as one of those presented in FIGS. 1, 2, 3, and 4, contain multiple types of dent-like markers, for example, drawn from the six markers presented in FIGS. 7A-F. One advantage of this embodiment is that it can improve visibility of the probe under different conditions. One advantage of this embodiment is that the probe is more likely to reflect ultrasound waves back toward the ultrasound transceiver.

Referring to FIG. 8A, in accordance with the present invention, a probe 800 with echogenic markers 801 and 802 is presented. The probe 800 has a straight shaft and can be of the types presented in FIGS. 1, 2, 3, and 4. The markers 801 are on the tip of the probe 800. The markers 802 are on the shaft of the probe 800. The markers 802 can be positioned under electrical insulation on the shaft of the probe 800. The probe 800 can be a radiofrequency cannula. The probe 800 can be a radiofrequency electrode. The probe 800 can be a microwave antenna. The probe is placed in a biological tissue 815. The biological tissue 815 can be a living body. The biological tissue 815 can be the human body. The biological tissue 815 can be the spine of a human. The biological tissue 815 can be a limb of a human. The biological tissue 815 can incorporate a human organ, such as the liver, kidney, prostate, lung, spleen, and pancreas. The biological tissue 815 can be an internal part of the human body. The probe 800 can be placed in a living body as part of a medical procedure. The probe 800 can be directed at a structure within the body, such as a tumor, a painful nerve, or nervous tissue. An ultrasound transceiver 805 is placed on the surface of the biological tissue 815. The ultrasound probe 805 can be placed on the surface of the skin. The ultrasound probe can be placed on an internal surface within a living body in the course of a surgical procedure. The ultrasound probe 805 is directed at the probe 800 and emits bursts of sound waves into the tissue. The sound waves include beams 810, 811, and 812. Beam 810 is incident on the probe 800 at its distal end of its straight tip, at the distal end of the cluster of markers 801. Beam 811 is incident on the probe 800 at the proximal end of its straight tip, between the cluster of markers 801 and the cluster of markers 802. Beam 812 is incident on the probe 800 at the proximal end of the cluster of markers 802. An array of ultrasound beams are present between beams 810 and 811, and between 811 and 812, as is understood by one skilled in the art.

Referring to FIG. 8B, in accordance with the present invention, a probe 850 with echogenic markers 851 and 852 is presented. The elements in FIG. 8B are identical to those in FIG. 8A except that probe 851 has a bent tip, whereas probe 800 has a straight tip. The tip lengths of probes 800 and 850 are identical, and the extent of markers 801 and 851 are identical. The ultrasound probe 855 transmits ultrasound beams 860, 861, and 862 into bodily tissue 865, and beams 860, 861, and 862 are incident on the probe 850. An array of ultrasound beams are present between beams 860 and 861, and between 861 and 862, as is understood by one skilled in the art.

Referring to both FIGS. 8A and 8B, the angle of the proximal shaft of probe 850 relative to ultrasound probe 855 is the same as the angle of the proximal shaft of probe 800 relative to the ultrasound probe 805. Due to the curve tip of probe 850, the image of the tip of probe 850 is larger in the ultrasound image produced by ultrasound probe 855, than is the image of the tip of probe 800 in the ultrasound image produced by ultrasound probe 805. Due to the curve tip of probe 850, the image of the echogenic markers 851 on the tip of probe 850 is larger in the ultrasound image produced by ultrasound probe 855, than is the image of the echogenic markers 801 on the tip of probe 800 in the ultrasound image produced by ultrasound probe 805. One advantage of a probe with echogenic markers and a bent tip is that its tip can be rotated to produce a larger ultrasound image signature in an ultrasound image than a probe with echogenic markers a straight tip placed in the living body with the same proximal shaft trajectory relative to the ultrasound transceiver. The echogenic markers 851 on probe 850 are more perpendicular to the ultrasound beams 860, 861, 862 than are the echogenic markers 801 on probe 800 relative to ultrasound beams 810, 811, 812. One advantage of a probe with echogenic markers and a bent tip is that if its echogenic markers produce a stronger ultrasound signal when oriented more perpendicular to the ultrasound beams, said probe with the echogenic markers and a bent tip can be oriented so that its echogenic markers produce a stronger ultrasound signal than the echogenic markers would if the probe had a straight tip.

Referring to FIG. 9A, an ultrasound marker with distal surface 900 and proximal surface 905 is presented in a cross-sectional view like that of marker 500, 505 in FIG. 5. The ultrasound marker 900, 905 is incut into the wall 909 of the tip of a straight probe, of which only a short segment is shown, that can be one of the probes presented in FIGS. 1, 2, 3, and 4. Surface 906 is the outer surface of the probe. The probe is placed within a living body and the shaft of the probe is oriented at a steep angle relative to the incoming ultrasound beam 910. The width of the beam that contacts the distal marker surface 900 is small since the surface 906 blocks the ultrasound beam. The reflected beam 911 is not directed toward the ultrasound transceiver since the angle of incidence of beam 910 on the distal surface 900 is steep.

Referring to FIG. 9B, an ultrasound marker with distal surface 950 and proximal surface 955 is presented in a cross-sectional view like that of marker 500, 505 in FIG. 5. The ultrasound marker 950, 955 is incut into the wall 959 of the tip of a bent-tip probe, of which only a short segment is shown, that can be one of the probes presented in FIGS. 1, 2, 3, and 4. Surface 956 is the outer surface of the probe. The probe is placed within a living body and the shaft of the probe is oriented at the same steep angle relative to the incoming ultrasound beam 960 as is the shaft of the probe in FIG. 9A relative to incoming beam 910; however, due to the bend in the tip of the probe in FIG. 9B, the width of the beam that contacts the distal marker surface 950 is large since the surface 956 does not occlude the distal marker surface 950. The reflected beam 961 is direct toward the ultrasound transceiver because the surface 950 is substantially perpendicular to the incoming beam 960. One advantage of a probe with ultrasound-enhancing markers and a curved tip is that the ultrasound image of the probe can be improved for steep angles of placement. One advantage of a radiofrequency cannula with ultrasound-enhancing markers and a curved tip is that the ultrasound image of the cannula can be improved for steep angles of placement.

While various patents have been incorporated herein by reference, to the extent there is any inconsistency between incorporated material and that of the written specification, the written specification shall control. In addition, while the disclosure has been described in detail with respect to specific embodiments thereof, it will be apparent to those skilled in the art that various alterations, modifications and other changes may be made to the disclosure without departing from the spirit and scope of the present disclosure. It is therefore intended that the claims cover all such modifications, alterations and other changes encompassed by the appended claims.

1. A radiofrequency probe having a distal end, a proximal end, a shaft, and an echogenic feature in the form of one or more indentations on the shaft comprising a distal surface and a proximal surface, wherein an angle between the distal surface and an outer surface of the shaft is smaller than an angle between the proximal surface and the outer surface of the shaft.

2. The probe of claim 1, wherein the probe has a curved tip.

3. The probe of claim 1, wherein the probe is a cannula.

4. The probe of claim 1, wherein the probe is an electrode.

5. The probe of claim 4, wherein the electrode is a unitized injection electrode.

6. The probe of claim 1, wherein the probe is tissue-piercing.

7. The probe of claim 1, wherein the shaft is a stiff shaft.

8. The probe of claim 1, wherein the shaft is composed of metal.

9. The probe of claim 1, wherein the probe is a radiofrequency cannula with a bevel configured for placement in an epidural space.

10. The probe of claim 1, wherein the probe is a needle configured to introduce a catheter.

11. The probe of claim 1, wherein the one or more indentations comprise a first indentation and a second indentation, wherein the distal surface of the first indentation has a first angle relative to the outer surface of the shaft, and the distal surface of the second indentation has a second angle relative to the outer surface of the shaft.

12. A needle having a distal end, a proximal end, a shaft, a curved tip and an echogenic feature in the form of one or more indentations on the shaft comprising a distal surface and a proximal surface, wherein an angle between the distal surface and an outer surface of the shaft is smaller than an angle between the proximal surface and the outer surface of the shaft.

13. The needle of claim 12, wherein the shaft is composed of metal.

14. The needle of claim 12, wherein the needle is a radiofrequency cannula.

15. The needle of claim 12, wherein the needle is part of a unitized radiofrequency electrode.

16. The needle of claim 12, wherein the needle is an epidural needle.

17. The needle of claim 12, wherein the needle is configured for effecting a nerve block.

18. The needle of claim 12, wherein the needle is a spinal needle.

19. The needle of claim 12, wherein the one or more indentations comprise a first and a second indentation, wherein the distal surface of the first indentation has a first angle relative to the outer surface of the shaft, and the distal surface of the second indentation has a second angle relative to the surface of the shaft.

20. A medical probe having a first echogenic feature and a second echogenic feature, wherein the first echogenic feature is an indentation in a surface of the probe, the second echogenic feature is a roughing of the surface of the probe, wherein the first echogenic feature and the second echogenic feature are in the same surface location on the shaft.

21. The medical probe of claim 20, wherein the probe is a needle, an epidural needle, a radiofrequency needle, a radiofrequency cannula, a radiofrequency electrode, an internally-cooled radiofrequency electrode, or a biopsy needle.

22. The medical probe of claim 20, wherein the probe has a curved tip.

23. The medical probe of claim 20, wherein the probe has a sharp bevel.

24. The medical probe of claim 20, wherein the probe has a blunt tip.

25. The medical probe of claim 20, wherein the roughing of the surface of the probe is produced by sandblasting.

26. The medical probe of claim 20, wherein the roughing of the surface of the probe is produced by beadblasting.

27. The medical probe of claim 20, wherein the indentation has a three-sided pyramidal shape.

28. The medical probe of claim 20, wherein the probe includes a shaft having a multitude of echogenic indentations.

29. (canceled)
